(19) United States
(12) Patent Application Publication
(10) Pub. No.: US 2011/0016579 A1
(43) Pub. Date: Jan. 20, 2011

(54) Gene of transporter selective to mugineic acid-iron complex

(75) Inventors: Yoshiko Murata, Osaka (JP); Takashi Iwashita, Osaka (JP)

Correspondence Address: DRINKER BIDDLE & REATH (DC), 1500 K STREET, N.W., SUITE 1100, WASHINGTON, DC, 20005-1209, US

(73) Assignees: SUNTORY LIMITED, Osaka-shi, Osaka (JP); SUNTORY HOLDINGS LIMITED, Osaka-shi, Osaka (JP)

(21) Appl. No.: 11/920,841

(22) PCT Filed: Nov. 24, 2005
(86) PCT No.: PCT/JP2005/021558

§ 371 Date: May 30, 2008

(30) Foreign Application Priority Data
May 24, 2005 (JP) 2005-151676

(51) Int. Cl. A01H 1/06; C07H 21/04; C12N 15/63; C12N 5/10; A01H 5/00; C12P 21/02; C07K 2/00; C07K 14/415; C07H 21/02

(52) U.S. Cl. 800/278; 536/23.1; 536/23.6; 435/320.1; 435/419; 800/295; 435/69.1; 530/350; 530/372; 800/320; 800/317; 800/298

(57) ABSTRACT

The invention provides a method for creating a transgenic plant comprising a gene containing a DNA to encode a transporter protein which selectively absorbs mugineic acid-iron complex. The transgenic plant is useful as a plant capable of growing in alkaline soil containing no bivalent iron but containing, for example, trivalent iron.

### TECHNICAL FIELD

The invention relates to a transporter protein from barley responsible for absorption of mugineic acid-iron complex from the soil, a gene encoding the protein, a vector containing the gene, and a transgenic plant using the vector.

### BACKGROUND ART

The proportion of the farmland capable of producing grains or tubers as staple foods is only about 10% of the total area of the land on the earth, and the remaining, about 90%, has been considered to be poor land to inhibit the growth of plants since it is quantitatively or qualitatively deficient in elements essential for the growth of plants. Since iron is a rate-determining factor for photosynthesis of the plants, in particular, the plants grown on a soil qualitatively or quantitatively deficient in iron develop iron-deficiency chlorosis and become destroyed. About 30% of the poor land is alkaline land where iron exists as trivalent iron, which is insoluble in water and therefore can hardly be absorbed by the plants through their roots. Accordingly, even when iron is abundant in the soil, the iron requirement for the healthy growth of the plants is not satisfied.

Gramineous plants secrete mugineic acid, a phytosiderophore (an iron chelator), into the soil when deficient in iron. It is thought that mugineic acid forms a complex with trivalent iron in alkaline soil, and that a transporter of the gramineous plant absorbs iron as mugineic acid-iron complex through roots thereof. Various studies have been carried out for elucidating the function of gramineous plants, and separation of the gene related to the phytosiderophore and a variety of transgenic plants with the gene introduced to them have been proposed. For example, a 36 kDa protein which is deeply involved in an iron acquisition mechanism via mugineic acid and improves iron absorption of gramineous plants, and a gene which encodes the protein have been elucidated (see patent document 1). It has been shown that the 36 kDa protein has a function as genes of a group of enzymes involved in the synthesis of mugineic acid.

Also, a gene IDS3 of an enzyme for biosynthesizing mugineic acid from deoxymugineic acid has been introduced into rice plant to enable the plant to secrete mugineic acid (see non-patent document 1), and a gene that encodes nicotianamine-aminotransferase (NAAT), an enzyme in the same biosynthesis path of mugineic acid is introduced into the rice plant to produce rice plant with improved iron-deficiency resistance (see patent document 2).

The maize yellow stripe 1 gene (ys1 gene), which encodes a membrane protein that mediates absorption of chelated iron from soil, has been cloned, and yellow stripe 1 protein (YS1 protein) has been isolated. It has also been elucidated that yeast and oocyte transformed with the gene that expresses YS1 protein is able to mediate non-selective absorption of metals, or absorption of other metals including heavy metals other than iron, for example copper, zinc, lead, cobalt or nickel (see patent document 3 and non-patent document 2). The YS1 protein is also reported to transport nicotianamine-iron complex involved in iron transport in plant cells (see non-patent documents 3 and 4).

Genes having homology as high as approximately from 70 to 80% with the gene that encodes YS1 protein have been found in rice plant (Oriza sativa; 14 genes) and thale cress (Arabidopsis thaliana; 8 genes). Of them, OsYSL2 of rice plant (see, for example, non-patent document 5) and AtYSL2 of thale cress (see, for example, patent document 3 and non-patent document 6) are reported to transport only nicotianamine iron complex without transporting mugineic acid-iron complex, and to be involved in iron transport in the plants. However, it has also been known that iron absorbed and transferred to stems and blades as nicotianamine iron complex is less than that as mugineic acid-iron complex (see patent document 4).

Although mugineic acid-iron complex is considered to be absorbed by the plant via a transporter specific to the complex, transporter protein that selectively absorbs mugineic acid-iron complex and the gene that encodes the protein have not been found yet.
Patent document 1: JP-A-2001-17181
Patent document 2: JP-A-2001-17012
Patent document 3: JP-T-2005-501502
Patent document 4: JP-A-2001-316192
Non-patent document 1: Kobayashi T. and five others, Planta 2001, vol. 212, pp. 864-871
Non-patent document 2: Curie, C. et al. , Nature 2001, vol. 49, p 346
Non-patent document 3: Schaaf. G. J. et al. , J. Biol. Chem. 2004, vol. 279, pp. 9091-9096
Non-patent document 4: Roberts, L. A. et al., Plant Physiol. 2004, vol. 135, pp. 112-120
Non-patent document 5: Koike, S. et al. , Plant J., 2004, vol. 39, pp. 415-424
Non-patent document 6: DiDonato, R. J. J. et al, Plant J. 2004, vol. 39, pp. 403-414

### SUMMARY OF THE INVENTION

The invention has an object to provide a method for cloning a gene for selectively absorbing mugineic acid-iron complex from soil into preferably an iron-deficient barley (Hordeum vulgare L.) through roots thereof and to transport it, and for creating a transgenic plant, to which the gene has been introduced, that can be raised in an iron-deficient state (alkaline soil) in the presence of mugineic acid.

The iron-acquisition mechanism of gramineous plants is comprised of synthesis of mugineic acid in the plants, release of the compound into soil, and absorption of mugineic acid-iron complex formed there by the plant. It is believed that, among gramineous plants, barley secretes the most mugineic acid and accordingly has the strongest alkali resistance. Therefore, plants other than barley that can actively grow like barley in alkaline soil can be developed provided that the transporter gene that helps absorption of mugineic acid-iron complex from soil by the plants is introduced into the plants other than barley.

The inventors have attempted to isolate the transport gene by extracting RNAs (using a kit manufactured by Invitrogen Co.) from a root of barley (Hordeum vulgare L.) grown in an iron-deficient state. Homology with the maize yellow (ZmYS1) gene was retrieved from the database of barley (DDBJ), and several ESTs having 60% or more of homology were found. Primers were formed based on the sequences of these ESTs, and were amplified with the said RNAs extracted from barley using 5′-, 3′-RACE (System of Rapid Amplification of cDNA Ends) (by Invitrogen Co. and Roche Co.) to isolate the transporter gene of barley with a total length of 2430 bp. The inventors have completed this invention through further studies thereafter.

That is to say, the present invention relates to:

(1) a gene containing a DNA encoding transporter protein for selectively absorbing mugineic acid-iron complex;

(2) the gene according to the above-mentioned (1), which is any one of (a) to (d) below:

(a) a gene comprising a DNA encoding a transporter protein having the amino acid sequence represented by SEQ ID NO: 2 in the sequence table;

(b) a gene comprising a DNA encoding a transporter protein having an amino acid sequence resulting from deletion, substitution, or addition of one or several amino acids in the amino acid sequence in (a), and having an activity for selectively absorbing mugineic acid-iron complex;

(c) a gene comprising a DNA encoding a transporter protein having an amino acid sequence of which homology with the amino acid sequence in (a) is at least 60%, and having an activity for selectively absorbing mugineic acid-iron complex; and

(d) a gene comprising a DNA that hybridizes with the DNA in (a) under a stringent condition and encodes a transporter gene having an activity for selectively absorbing mugineic acid-iron complex;

(3) a vector characterized by containing the gene according to the above-mentioned (1) or (2);

(4) a host cell characterized by containing the vector according to the above-mentioned (3);

(5) a transgenic plant into which the gene according to the above-mentioned (1) or (2) is introduced;

(6) a transgenic plant into which the vector according to the above-mentioned (3) is introduced;

(7) a method for producing a transporter protein having an activity for selectively absorbing mugineic acid-iron complex, characterized by cultivating the host cell according to the above-mentioned (4) under a condition for expressing the gene according to the above-mentioned (2);

(8) a transporter protein having an activity for selectively absorbing mugineic acid-iron complex and being produced by the method according to the above-mentioned (7);

(9) a protein, which is any one of (a) to (c) below having an activity for selectively absorbing mugineic acid-iron complex:

(a) a protein comprising an amino acid sequence represented by SEQ ID NO: 2 in the sequence table;

(b) a protein comprising an amino acid sequence resulting from deletion, substitution, or addition of one or several amino acids in the amino acid sequence in (a), and having an activity for selectively absorbing mugineic acid-iron complex; and

(c) a protein comprising an amino acid sequence of which homology with the amino acid sequence in (a) is at least 60%, and having an activity for selectively absorbing mugineic acid-iron complex;

(10) the RNA transcript of the DNA according to the above-mentioned (1);

(11) the transgenic plant according to the above-mentioned (6) characterized by belonging to any family selected from the group consisting of Poaceae, Moraceae, Leguminosae, Rosaceae, Theaceae, Rubiaceae, Fagaceae, Rutaceae and Solanaceae; and

(12) a method for giving an activity for selectively absorbing mugineic acid-iron complex to a plant characterized by permitting the gene according to the above-mentioned (1) or (2) to be expressed in the plant.

The invention provides a transporter gene HvYS1 (Hordeum Vulgare Yellow Stripe 1) that helps selective absorption of mugineic acid-iron complex, preferably identified from barley that is the most resistant to iron deficiency among the gramineous plants and is capable of absorbing trivalent iron ions into the plant even in alkaline soil, and transporter protein thereof. By taking advantage of the transporter gene and the mechanism of absorbing mugineic acid-iron complex, transgenic plants (for example crops) capable of growing in alkaline soil, in which such plants have not been able to grow, may be developed. Since the transgenic plants can grow in alkaline soil containing no divalent iron but containing, for example, trivalent iron, even a poor land, particularly alkaline soil that has not been suitable for a farm may be utilized as a farm. This means that planting area for staple food plants such as crops and vegetables may be expanded so as to be sufficient for supplementing food shortage due to increasing population.

Also, the invention may be used for expanding dairy land because meadows may be expanded by introducing the gene of the invention into grasses.

Since the transgenic plants of the invention have a function for selectively absorbing mugineic acid-iron complex, unlike the plants into which a transporter gene that allows non-selective absorption of metals into the plant has been introduced, there is smaller risk of absorbing metals other than iron, for example, heavy metals harmful to the human body. Accordingly, crops that are safe as food may be produced.

The transgenic plants of the invention are characterized by rapid growth since iron necessary for photosynthesis may be absorbed even when cultivated in alkaline soil. Consequently, productivity of plants other than barley may be enhanced by introducing the transporter gene of the invention into the plants.

Bacteria, yeast, animal cells or plant cells that have been transformed by introduction of the transporter gene of the invention may be used as cells for elucidating the transporter mechanism. In addition, the transporter gene of the invention and partial base sequences thereof may be used as probes for other transporter genes.

### BRIEF DESCRIPTION OF THE DRAWINGS

FIG. 1 shows an amino acid sequence determined from cDNA of HvYS1 in barley. Boxes show homology of the sequence with ZmYS1 in maize, and Roman numerals denote membrane-spanning regions of ZmYS1 (12 regions) predicted by the SOSUI program.

FIG. 2 is a drawing of HvYS1 expression in barley tissue.

FIG. 3 shows the results of Example 3. HvYS1 denotes an HvYS1-expressing DDY4 strain, ZmYS1 denotes a ZmYS1-expressing DDY4 strain, and VEC denotes a DDY4 strain into which only the vector has been introduced. Fe(III)-citrate denotes iron(III) complexed with citrate, Fe(III)-MA denotes iron(III) complexed with mugineic acid-iron(III) complex, and Fe(II)-NA denotes iron(II) complexed with nicotianamine.

FIG. 4 shows the electrophysiological responsiveness in the HvYS1-expression oocyte cells of Xenopus to various mugineic acid-metal complexes and nicotinamide-iron(II) complex. The vertical axis represents rates of voltage changes (%) of other metal complexes assuming the voltage change of mugineic acid-Fe(II) complex to be 100%.

FIG. 5 shows localization of HvYS1 in a root of iron-deficient barley. In the drawing, a and b denote vertical cross sections of the root, while c and d denote transverse cross sections of the root. a and c show the results of hybridization with a sense probe (negative control), and b and d show the results of hybridization with an antisense probe. Scale: 100 μm.

FIG. 6 is a schematic illustration of a plasmid Mac-HvYS1-mas-pBinPlus.

FIG. 7 shows HvYS1 expression by RT-PCR in transgenic plants. In the drawing, 1, 2 and 3 denote HvYS1-expressing transgenic plants, and 4 and 5 denote usual plants (negative controls) into which HvYS1 is not introduced. M denotes a molecular weight marker.

### DESCRIPTION OF THE PREFERRED EMBODIMENTS

“Mugineic acid-iron complex” refers to a chelate compound resulting from coordinate bond of mugineic acid with iron ions, especially trivalent iron ions. Examples of mugineic acid include mugineic acid, 2′-deoxymugineic acid, 3-hydroxymugineic acid, 3-epihydroxymugineic acid, avenic acid, distichonic acid and epihydroxydeoxymugineic acid. The phrase “To selectively absorb mugineic acid-iron complex” refers to transferring and transporting only mugineic acid-iron complex from the outside to the inside of cells, and not transferring and transporting complex compounds formed between mugineic acid and metals other than iron, or chelate complex compounds formed by coordination of mugineic acid analogues, for example, nicotianamine, with iron ions.

While the “transporter protein” refers to a protein on cell membrane that is responsible for transport of substances through the membrane, the term in this specification means a protein responsible for transport of mugineic acid-iron complex through the cell membrane. The protein preferably has an activity for selectively absorbing mugineic acid-iron complex.

An example of the protein having an activity for selectively absorbing mugineic acid-iron complex is the protein having the amino acid sequence represented by SEQ ID NO: 2. Proteins resulting from deletion, substitution or addition of one or plural amino acids in the protein having the amino acid sequence represented by SEQ ID NO: 2 may be included in the proteins according to the invention, as far as the protein has a function for exhibiting an activity for selectively absorbing a mugineic acid-iron complex. The said “plural” preferably refers to 20 or less, more preferably 10 or less, and further preferably 5 or less. The phrase “deletion, substitution or addition of one or plural amino acids” in the amino acid sequence as used herein refers to deletion, substitution or addition of amino acids as a result of known technical methods such as gene engineering or site-specific mutagenesis, or natural phenomenon.

Also, a protein having at least 60% or more, preferably 70% or more, more preferably 80% or more, further preferably 90% or more, and particularly preferably 95% or more of homology with the above-mentioned amino acid sequence may be included in the protein according to the invention, as far as the protein has an activity for selectively absorbing mugineic acid-iron complex. “Homology” of the amino acid sequence refers to the extent of matching of amino acid residues that constitute respective sequences in the comparison of the primary structure between proteins.

The “gene” means a functional unit of DNA, and bears specific information on proteins. The gene that contains the DNA encoding the transporter protein in this specification (maybe abbreviated as a transporter gene in the specification) has information on the transporter protein having an activity for selectively absorbing mugineic acid-iron complex. Accordingly, the transporter gene includes a DNA sequence that encodes the transporter protein and/or a regulatory sequence necessary for expression of the gene, but is not limited to them. The transporter gene may also include, for example, non-expressing DNA segments that form recognition sequences for other proteins.

Examples of RNA transcripts include primary transcripts of DNA that encodes the transporter protein, and mature mRNA, tRNA and rRNA functionalized by precursor RNA chain cleavage, 3′-end formation, RNA splicing or RNA editing by post-transcription processing.

To obtain the transporter gene of the invention, for example, mRNA is extracted from a source of mRNA that encodes the transporter protein, and cDNA is prepared using a reverse transcriptase. Then, for example, 3′-RACE (Rapid Amplification of cDNA Ends), 5′-RACE, and/or 5′/3′-RACE is applied in order to obtain the desired transporter gene. To design primers used for 3′-RACE, 5′-Race and/or 5′/3′-RACE, it is preferable that homology retrieval from the database of barley based on the known gene encoding the membrane protein that mediates absorption of chelated iron is carried out, ESTs that exhibit 60% or more of homology with the known gene are selected from the gene sequence of barley, and the obtained ESTs are used for the designing.

Examples of the source of mRNAs that encode the transporter protein include gramineous plants cultivated hydroponically such as barley, wheat, rye, oats, maize, sorghum and rice, and the roots of barley may be preferably used. Since the transporter gene of the invention is expressed in an iron-deficient environment, the roots of gramineous plants (preferably barley) exposed to an iron-ion-free environment or an alkaline environment in which iron ions exist as trivalent ions insoluble in water may be favorably used. Alternatively, a gramineous plant (preferably barley) may be seeded on a solid medium such as a GM medium or Murashige & Skoog medium (hereinafter called MS medium), and the roots of the gramineous plant (preferably barley) grown under an aseptic condition may be used. The source may be a callus or cultivated cells of a gramineous crop (preferably barley) grown under an aseptic condition, and any source may be used as far as the cell contains the mRNA of the desired gene.

mRNA may be extracted from a mRNA source by known methods. For example, the plant of barley grown in hydroponic culture is exposed to an iron-deficient condition, followed by sampling the root. The sampled root is frozen with liquid nitrogen, and then mashed in a mortar or the like. While mRNA may be extracted from the mashed root using a glyoxal method, guanidine thiocyanate-cesium chloride method, lithium chloride-urea method, proteinase K-deoxyribonuclease method, or AGPC (Acid Guanidinium-Phenol-Chloroform) method, a commercially-available RNA-extraction kit may be used for extraction. Examples of the commercially-available RNA-extraction kit include RNA isolation kit (by Stratagene Co.), Isogene (Nippon Gene Co.), Trizol (by Invitrogen Co.), and RNA extraction reagent for concert plants (by Invitrogen Co.). The extraction should be performed in accordance with the manual of each kit. mRNAs may be purified with a column (for example RNeasy by QUIAGEN Co.) after extraction.

The said 3′-RACE may be implemented using a commercially-available kit, for example, 3′-RACE (System of Rapid Amplification of cDNA Ends; by Invitrogen Co.), 3′RACE System for Rapid Amplification of cDNA Ends (by Life Technologies Co.), or 3′-full RACE core set (by Takara Bio Inc.).

The said 5′-RACE may be implemented using a commercially-available kit, for example, 5′-RACE (by Invitrogen Co.), Cap Fishing Full-Length cDNA Premix kit (by Funakoshi Corp.), or 5′-full RACE core set (by Takara Bio Inc.). 5′/3′ may be implemented using 5′/3′-RACE kit, 2^{nd} generation (by Roche Co.), or the like.

The primer used for 3′-RACE, 5′-RACE or 5′/3′-RACE is preferably an oligonucleotide having about 15 to 25 by of a nucleotide sequence with 90% or more, preferably 95% or more, and more preferably 98% or more of homology with the partial nucleotide sequence of the gene that encodes the membrane protein for mediating absorption of known iron chelate compounds. Examples of the primer for 3′-RACE include oligonucleotides having base sequences represented, for example, by SEQ ID NO: 4, 5, 6, or 7. Examples of the primer for 5′-RACE include oligonucleotides having base sequences represented for example, by SEQ ID NO: 8, 9, 10, 11, or 12. Examples of the primer for 5′/3′-RACE include oligonucleotides having base sequences represented, for example, by SEQ ID NO: 14 or 15.

Known genes that encode the membrane protein for mediating absorption of known chelate iron compounds, for example, maize yellow stripe 1 gene (SEQ ID NO: 3) deposited with Accession Number AF 186234 of GenBank, may be preferably used.

Examples of the above EST include sequences deposited with Accession Number AF472629, BJ470821, BJ448359, or BQ765689 in DDBJ of barley. EST is a gene fragment with a sequence determined from the 3′-end or 5′-end of complementary DNA (cDNA) clone, and usually has a length of from 300 to 400 nucleotides.

The above-mentioned homology retrieval may be performed in databases such as GenBank or DDBJ using analysis software such as BLAST and FASTA. EST to be retrieved is preferably a gene having particularly high homology in an amino acid sequence in a highly conservative region or in a region supposed to have functions. The sequence preferably conserves amino acids essential for the function of the protein.

PCR may be performed by known methods. The PCR product may be inserted into a vector, introduced into a host and amplified.

The entire base sequence of the obtained gene may be determined by known methods. While examples of the method for determining the base sequence include the Maxam-Gilbert chemical modification method and a dideoxynucleotide strand termination method using M13 phage, the nucleotide sequence is usually determined using an automatic sequencer (for example automatic DNA sequencer ABI PRISM M™ 310 Genetic Analyzer by Perkin Elmer Japan).

A gene having the base sequence represented by SEQ ID NO: 1 in the sequence table may be thus isolated, for example, as the gene containing the DNA (nucleotide sequence 169 to 2202 in SEQ ID NO: 1 of the sequence table) that encodes the transporter protein.

The DNA also include a DNA that hybridizes under a stringent condition with a DNA having a complementary base sequence to the DNA that encodes the transporter protein, and has an activity for selectively absorbing mugineic acid-iron complex. The “DNA that hybridizes under a stringent condition” refers to, for example, a DNA obtained by a colony hybridization method, a plaque hybridization method, or a southern blot hybridization method using a partial sequence of the DNA encoding the transporter protein having the amino acid sequence represented by SEQ ID NO: 1 as a probe. The “stringent condition” as used herein refers to a condition in which DNAs having at least about 50% or more, preferably about 60% or more and more preferably about 80% or more of homology with the base sequence represented by SEQ ID NO: 1 hybridize with each other, but DNAs having lower homology do not hybridize with each other; or a condition in which DNAs hybridize with each other in a SCC solution having from about 0.1 to 2 times of concentration (the composition of the SCC solution having 1 time of concentration comprises 150 mM of sodium chloride and 15 mM of sodium citrate) at a temperature of about 65° C.

“DNA” as used herein refers to deoxyribonucleic acid. The unit of DNA is referred to as a nucleotide, and is composed of a base, sugar (D-deoxyribose), and phosphoric acid. There are 4 kinds of bases, adenine (A), guanine (G), cytosine (C) and thymine (T), and genetic information is determined by the arrangement of these four bases.

Once the base sequence has been determined, the transporter gene of the invention may be obtained thereafter by chemical synthesis, by PCR using the cDNA or genome DNA of the gene as a template, or by hybridizing DNA fragments having the corresponding nucleotide sequences as probes.

In addition, the transporter gene of the invention contains a DNA that encodes a protein having the amino acid sequence represented by SEQ ID NO: 2. Genes that encode proteins having the amino acid sequence represented by SEQ ID NO: 2 in which one or plural amino acids are deleted, substituted or added may also be included in the transporter gene of the invention, as far as the proteins have a function that exhibits an activity for selectively absorbing mugineic acid-iron complex. The phrase “deletion, substitution or addition of one or plural amino acids” means the same as in the above description of protein. Mutation may be introduced into the transporter gene of the invention by a known method such as the Kunkel method or the Gapped duplex method or a similar method using, for example, a mutagenesis kit (for example, Mutant-K or Mutant-G by Takara Bio Inc.) employing a site-directed mutagenesis method, or using LA PCR in vitro Mutagenesis series kit (by Takara Bio Inc.).

Genes that encode proteins having at least 60% or more, preferably 70% or more, more preferably 80% or more, further preferably 90% or more, and particularly preferably 95% or more of homology with the above-mentioned amino acid sequence may also be included in the transporter gene of the invention, as far as the proteins have a function that exhibits an activity for selectively absorbing mugineic acid-iron complex.

“Homology” regarding the above-mentioned amino acid sequence means the same as in the above description of protein.

The activity of the transporter protein according to the invention for selectively absorbing mugineic acid-iron complex may be confirmed, for example, by transforming a double mutant fet3fet4 (DDY4 strain) of budding yeast Saccharomyces cerevisiae by introducing the transporter gene of the invention, and by cultivating the transformed yeast in a medium supplemented with mugineic acid-iron(III) complex. Since DDY4 strain is an yeast that is defective in the divalent iron-absorption system, is unable to grow in an iron-limiting medium (Eide, D. et al., Proc. Natl. Acad. Sci. USA, 1996, vol. 93, pp. 5624-5628) and is unable to grow by taking advantage of mugineic acid-iron (III) complex (Loulergue, C., Gene 1998, vol. 225, pp. 47-57), the yeast having an activity power for selectively absorbing mugineic acid-iron complex can grow on a medium supplemented with mugineic acid-iron(III) complex but the yeast having no above-mentioned activity power cannot grow on the medium.

The activity power for selectively absorbing mugineic acid-iron complex may be also confirmed by observing cell membrane voltage changes using Xenopus oocyte cells. The voltage change of the cell membrane may be obtained by directly measuring the voltage difference between the inside and outside of the cell membrane by, for example, a membrane voltage clamp method, wherein the voltage change of the oocyte cell membrane occurs in accordance with the absorption of mugineic acid-iron complex via the transporter protein expressed in the oocyte cell after adding a solution containing mugineic acid-iron complex to the oocyte cell into which the transporter gene of the invention has been introduced.

The transporter protein according to the invention may be obtained by introducing the transporter gene of the invention into a vector, cultivating a host transformed with the vector under an inducing condition, and purifying the protein from the host.

The term “vector” refers to a substance that functions for introducing a gene into a cell, and examples of the vector include plasmids, viral vectors, and artificial non-viral vectors. While examples of the non-viral vector include liposomes and polylysine compounds, they are not limited thereto.

The vector according to the invention may be constructed by integrating the transporter gene of the invention, a promoter and a terminator into a multi-cloning site of a vector that serves as a base (referred to a basic vector hereinafter for the convenience of descriptions). The basic vector is not particularly limited as far as it is replicable in a host, and examples of the basic vector include plasmid DNA and phage DNA. Examples of the plasmid DNA include E. coli host plasmids such as pBR322, pBR325, pUC118, and pUC119; B. subtilis host plasmids such as pUB110 and pTP5; yeast host plasmids such as pFL61 (by ATCC Co.), YEp13, YEp24 and YCp 50; plant cell host plasmids such as pUC plasmids (pUC18, pUC19, PSR-01,PSA-01, PSR-02, and PSR-03 by Kumiai Chemical Industry Co., Ltd.) and pBI221; and binary vectors such as pWTT23132 (by DNAP Co.). Examples of the phage DNA include λ-phage. Animal viruses such as retro virus and vaccinia virus, and insect viruses such as Baculo virus may also be used.

The vector is not particularly limited as far as it is a plant-cell-host vector capable of transforming the plant when a transgenic plant is produced by introducing the transporter gene of the invention.

Any promoters capable of being expressed in the host may be used. For example, when the host is E. coli, preferable promoters are E. coli-derived promoters such as trp promoter, lac promoter, P_{L} promoter, and P_{R} promoter. When the host is B. subtilis, preferable promoters are SPO1 promoter, SPO2 promoter, and penP promoter. When the host is yeast, preferable promoters are pFL61 promoter (by ATCC Co.), PHO5 promoter, PGK promoter, GAP promoter, and ADH promoter. When the host is a plant, preferable promoters are plant-derived promoters such as 35S RNA promoter of cauliflower mosaic virus, rd29A gene promoter, and rbcS promoter, and constitutive promoters such as mac-1 promoter produced by adding the enhancer sequence of the cauliflower mosaic virus 35S promoter to the 5′ side of the mannopine-synthetase-promotor sequence derived from Agrobacterium. Artificially designed and modified promoters such as tac promoter may be used, and mac-1 promoter is preferable among them. When a vector constituted using the said mac-1 promoter is inserted into the genome of a plant, the gene (HvYS1) linked downstream of the promoter may be expressed at a high level in almost all the organs of the plant in any stage of growth.

Any terminators capable of being expressed in the host may be used. Examples of the terminator when the host is a plant include rrn terminator, psbA terminator, 35S terminator, rps16 terminator, CaMV35S terminator, ORF25polyA transcription terminator, and PsbA terminator.

The vector according to the invention preferably has a gene for discriminating gene recombinants. The gene for discriminating the gene recombinant is not particularly limited, and any known genes, per se, may be used. Examples of the gene include various drug-resistant genes and genes for complementing auxotrophy of the host. More specifically, examples of the gene include ampicillin-resistant gene, neomycin-resistant gene (G418 resistant), chloramphenicol-resistant gene, kanamycine-resistant gene, spectinomycin-resistant gene, URA3 gene, tetracycline-resistant gene, and chlorsulfuron (herbicide)-resistant gene. The gene preferably has a promoter and a terminator for discriminating the gene at the upstream and downstream of the gene.

Other genes, for example a gene encoding mugineic acid biosynthetase, may be introduced into the vector according to the invention. When a gene encoding mugineic acid biosynthetase as well as the transporter gene of the invention are introduced into a vector, and a plant is transformed with the vector, the plant may be able to absorb mugineic acid-iron complex in alkaline soil containing no mugineic acid because the plant acquires not only the function for selectively absorbing mugineic acid-iron complex but also the ability to biosynthesize mugineic acid and secrete it into the soil. While examples of the gene that encodes mugineic acid biosynthetase include a gene that encodes 36 kDa protein described in JP-A-2001-17181, and a gene that encodes nicotianamine-amino group transferase described in JP-A-2001-17012, the gene is not limited thereto. The above-mentioned other genes include genes that are hybridized with the above-mentioned other genes under a stringent condition, and that are including DNAs encoding a protein that biosynthesize mugineic acid. The stringent condition is as described above.

The method for producing the vector according to the invention is not particularly limited. Segments of respective DNAs (promoter, terminator, transporter gene of the invention, and drug-resistant gene) may be introduced into the basic vector in a predetermined order.

The method for introducing the vector into the host is not particularly limited, and examples of the method include a method using calcium ions (Cohen, S. N. et al.: Proc. Natl. Acad. Sci., USA, vol. 69, pp. 2110-2114, 1972), an electroporation method (Becker, D. M. et al., Methods Enzymol., Vol. 194, pp. 182-187, 1990), a spheroplast method (Hinnen, A. et al.: Proc. Natl. Acad. Sci., USA, vol. 75, pp. 1929-1933, 1978), and a lithium acetate method (Itoh, H., J. Bacteriol., vol. 153, pp. 163-168, 1983). While there are various methods other than those described above such as a microinjection method, a micro-projectile bombardment method (also referred to a particle acceleration method or biolistic bombardment method), a transformation method with a virus, a transformation method with an agrobacterium, a particle gun method (Svab, Z., Hajdukiewicz, P. and Maliga, P., Proc. Natl. Acad. Sci., USA, 1990, vol. 87, pp. 8526-8530), and the PEG method (Golds, T., Maliga, P., and Koop, H-U., Bio/Technol., 1993, vol. 11, pp. 95-97), the method is not limited thereto.

The method for proliferating the host into which the vector according to the invention has been introduced is not particularly limited, and known methods may be preferably used depending on the host.

The transporter protein according to the invention may be separated from the host cell and purified by an appropriate combination of known separation and purification methods. Examples of these known separation and purification methods include a method taking advantage of solubility such as salting-out and solvent precipitation methods, a dialysis method, an ultrafiltration method, a gel filtration method, a method mainly taking advantage of the difference in molecular weights such as an SDS-polyacrylamide gel electrophoresis method, a method taking advantage of the difference in charges such as ion-exchange chromatography, a method taking advantage of specific affinity such as affinity chromatography, a method taking advantage of the difference in hydrophobicity such as reversed-phase high-performance liquid chromatography, and a method taking advantage of the difference in isoelectric point such as an isoelectric point electrophoresis method.

Operations of the above-mentioned gene-engineering and bioengineering methods may be readily implemented according to the methods described in commercially available handbooks of experiments such as Molecular Cloning by Cold Spring Harbor Laboratory (published in 1982) and Molecular Cloning 2^{nd} Edition by Cold Spring Harbor Laboratory (published in 1989).

Transgenic plants in which the transporter gene of the invention is expressed or over-expressed may be created by using the above-mentioned gene manipulation methods. While the transgenic plant according to the invention produces the transporter protein by the expression of the transporter gene of the invention, the transporter gene is preferably expressed in epidermal cells of the roots. Absorption of mugineic acid-iron complex in soil may be facilitated by permitting the transporter gene of the invention to be expressed on the surfaces of the roots. Expression of the gene in the transgenic plant may be confirmed by histological staining, which may be implemented by known methods.

The transgenic plant of the invention may be cultivated in soil containing no divalent iron, for example in alkaline soil containing trivalent iron and mugineic acid-iron complex. Since the transgenic plant of the invention absorbs iron necessary for photosynthesis, the plant is characterized in rapid growth, and consequently productivity of the plant may be improved.

Monocotyledonous plants and dicotyledonous plants are preferable as the plant transformed by using the transporter gene of the invention. More specifically, examples of the plant include Poaceae (such as rice, barley, wheat, oats, rye, maize, millet, barnyard millet, kaoliang, and pasturage), Moraceae (such as mulberry, hop, paper mulberry, gum tree, and hemp), Leguminosae (such as soy bean, red bean, peanut, kidney bean, and horse bean), Rosaceae (such as strawberry, ume tree, and rose), Theaceae (such as tea tree), Rubiaceae (such as coffee tree and gardenia), Fagaceae (such as Japanese oak, beech, and oak), Rutaceae (such as sour orange, yuzu orange, unshu orange, and Japanese pepper), and Solanaceae (such as eggplant, tomato, red pepper, potato, tobacco plant, hairy thorn apple, ground cherry, petunia, calibrachoa, and Nierembergia). However, the plant is not limited thereto.

While the invention is described in more detail with reference to examples, the invention is not limited to these examples. “%” denotes % by volume, unless otherwise stated. Abbreviations in the specification are as follows.
a: adenine
c: cytosine
g: guanine
t: thymine
PBS: phosphate buffer saline
PCR: polymerase chain reaction
RACE: rapid amplification of cDNA ends
EST: expressed sequence tag
RT-PCR: reverse transcription-polymerase chain reaction
SOSUI: secondary structure presumption system of membrane protein
Fe(III).citrate: citric acid-iron complex (iron-ammonium citrate complex)
Fe(III).MA: mugineic acid-iron complex (mugineic acid-iron(III) complex)
Fe(II).NA: nicotianamine-iron complex
Tris: tris(hydroxymethyl)aminomethane
EDTA: ethylenediamine tetraacetic acid
HEPES: 2-[4-(2-hydroxyethyl)-1-piperazinyl]ethane sulfonic acid
MAS coat: Matsunami adhesive slide glass
DIG: digoxigenin

### Example 1

Cloning of HvYS1 cDNA

### (1) Extraction of Total RNA

After seeding barley (Morex sp.), the seeds were cultivated in ⅕ Hoagland cultivation medium (hereinafter, referred to a cultivation medium). On day 16 from seeding, the young plants were subjected to iron-deficient treatment (cultivation in an iron-free cultivation medium) for 4 days. The roots of the plants were collected, and total RNA was extracted using the Concert Plant RNA Extraction Reagent (by Invitrogen Co.).

### (2) 3′-RACE

cDNA was synthesized from total RNA (1 μg) with reverse transcriptase. Obtained cDNA was amplified by 3′-RACE (System of Rapid Amplification of cDNA Ends by Invitrogen Co.). Four ESTs (AF472629, BJ470821, BJ448359, and BQ765689) having 60% or more of homology were detected in the database of barley (DDBJ) using ZmYS1 as retrieval sequences, the base sequences in Table 1 were selected from the sequence of BJ470821, and oligonucleotides synthesized from the sequences were used as the primers used for 3′-RACE.

TABLE 1

| Primer | Base sequence | Sequence table |
|---|---|---|
| 3′RACE-GSP | 5′-CATTGCCGGCCTTGTTGCT | SEQ ID NO: 4 |
| | G | |
| 3′RACE- | 5′-CGGCCTTGTTGCTGGCACC | SEQ ID NO: 5 |
| NestGSP | | |

cDNA obtained by 3′-RACE was developed by 1% (w/v) agarose gel electrophoresis, and was purified using Quiagen GIA Quick Gel Extraction Kit (by Quiagen Co.). The purified cDNA was inserted into pCRII-TOPO vector (4.0 kb) of TOPO TA Cloning Version R (manufactured by Invitrogen Co.), and E. coli TOP10 was transformed with the vector. Transformation products were amplified by colony PCR, and the base sequence of the product of a predicted length was determined with an automatic DNA sequencer (ABI PRISM™ 310 Genetic Analyzer, by Perkin Elmer Japan). The primers in Table 2 were used for the sequencer.

TABLE 2

| Primer | Base sequence | Sequence table |
|---|---|---|
| M13R | 5′-CAGGAAACAGCTATGAC | SEQ ID NO: 6 |
| M13F | 5′-GTAAAACGACGGCCAG | SEQ ID NO: 7 |

### (3) 5′-RACE

cDNA was synthesized from total RNA (1 μg) with reverse transcriptase as in 3′-RACE. Obtained cDNA was used for 5′-RACE (by Invitrogen Co.). The nucleotide sequences in Table 3 were selected from the sequence of AF472629 of ESTs detected in (2) above, and oligonucleotides synthesized from the sequence were used as the primers used for 5′-RACE.

TABLE 3

| | | Sequence |
|---|---|---|
| Primer | Base sequence | table |
| 5′RACE- | 5′-CCACAAGCATCGCCTCCAG | SEQ ID NO: 8 |
| GSP1 | | |
| 5′RACE- | 5′-CATCGCCTCCAGTGTAGAACC | SEQ ID NO: 9 |
| GSP2 | | |
| 5′RACE- | 5′-CAGTGTAGAACCATTGGAAG | SEQ ID NO: 10 |
| GSP3 | | |

cDNA obtained by 5′-RACE was developed by 1.2% (w/v) agarose gel electrophoresis. Extraction of the gene from the gel and transformation of E. coli by the gene were performed by the same methods as in the case of cDNA obtained by 3′-RACE in (2) above. Sequencing of the transformation product was the same as in 3′-RACE in (2) above, and the sequence at the 5′-side was partially determined.

Since the 5′-end has a higher-order structure, the sequence to the 5′-end was determined using 5′/3′-Race Kit, 2^{nd} Generation (by Roche Co.) by operating the same as in 5′-RACE, which contains mRNA reverse transcriptase having a higher optimum temperature (55° C.). The base sequences in Table 4 were selected from the sequence of AF472629, and oligonucleotides synthesized from the sequence were used as the primers used for 5′/3′-RACE.

TABLE 4

| | | Sequence |
|---|---|---|
| Primer | Base sequence | table |
| N5′RACE- | 5′-GAATAGCAGTTGCAGTCC | SEQ ID NO: 11 |
| GSP1 | | |
| N5′RACE- | 5′-GTAGTCGACGACCAGTACCTG | SEQ ID NO: 12 |
| GSP2 | | |
| N5′RACE- | 5′-CGACCAGTACCTGTCTCAGG | SEQ ID NO: 13 |
| GSP3 | | |

### (4) Confirmation of Nucleotide Sequence

cDNA obtained with the reverse transcriptase from total RNA obtained in (1) above was subjected to PCR in order to confirm the joint portion of the sequence determined by 5′/3′-RACE with the base sequence determined by 5′-RACE. A forward primer (SEQ ID NO: 14 in the sequence table) synthesized by selecting the base sequence in Table 5 from the sequence in AF47269 and a reverse primer (SEQ ID NO: 15 in the sequence table) synthesized by selecting the base sequence in Table 5 from the sequence BJ470821 were used for PCR.

TABLE 5

| Primer | Base sequence | Sequence table |
|---|---|---|
| Forward | 5′-GAATAATGAGGCCACTCATC | SEQ ID NO: 14 |
| primer | | |
| Reverse | 5′-GGCTATAACAACATAGTACC | SEQ ID NO: 15 |
| primer | | |

cDNA of the obtained PCR product was subjected to agarose gel electrophoresis, the gene was extracted from the gel, and E. coli was transformed by the gene using the same method as in (2) above to determine the total nucleotide sequence.

Since the total nucleotide sequence has been determined, the base sequence of the total length was subjected to PCR again using cDNA obtained from the total RNA of the roots of barley obtained in (1) above with reverse transcriptase and using the primers in Tables 6 and 7.

TABLE 6
1^{ST} PCR

| Primer | Base sequence | Sequence table |
|---|---|---|
| Forward | 5′-GCACACGGTTCCAGCTCGCC | SEQ ID NO: 16 |
| primer | | |
| Reverse | 5′-GATAGTTCAGCAAGGCACAAC | SEQ ID NO: 17 |
| primer | | |

TABLE 7
2^{ND} PCR

| Primer | Base sequence | Sequence table |
|---|---|---|
| Forward | 5′-CCTCCAGTGATTCTTCTTCC | SEQ ID NO: 18 |
| primer | | |
| Reverse | 5′-GATAGTTCAGCAAGGCACAAC | SEQ ID NO: 19 |
| primer | | |

1.2% (w/v) agarose gel electrophoresis was employed for the cDNA obtained by PCR, and extraction from the gel and transformation into E. coli were performed by the same method as in (2) above. Transformed E. coli was cultivated overnight at 37° C. on LB (Luria-Bertani) medium supplemented with 50 μg/mL of ampicillin, and DNA was extracted from the culture with Mini-M Plasmid DNA Extraction System (by VIOGENE Co.). The base sequence of this DNA was determined and confirmed (SEQ ID NO: 1), and the DNA was named HvYS1 (Hordeum Vulgare Yellow Stripel, DDBJ Accession No. AB214183).

The primers in Table 8 were used as the sequencing primers for base sequence determination.

TABLE 8

| Primer | Base sequence | Sequence table |
|---|---|---|
| M13R | 5′-CAGGAAACAGCTATGAC | SEQ ID NO: 6 |
| M13F | 5′-GTAAAACGACGGCCAG | SEQ ID NO: 7 |
| 5′M | 5′-CCTCCTCGCTTGCAGCTTCG | SEQ ID NO: 20 |
| 3′M | 5′-GGTGCCAGCAACAAGGCCGG | SEQ ID NO: 21 |

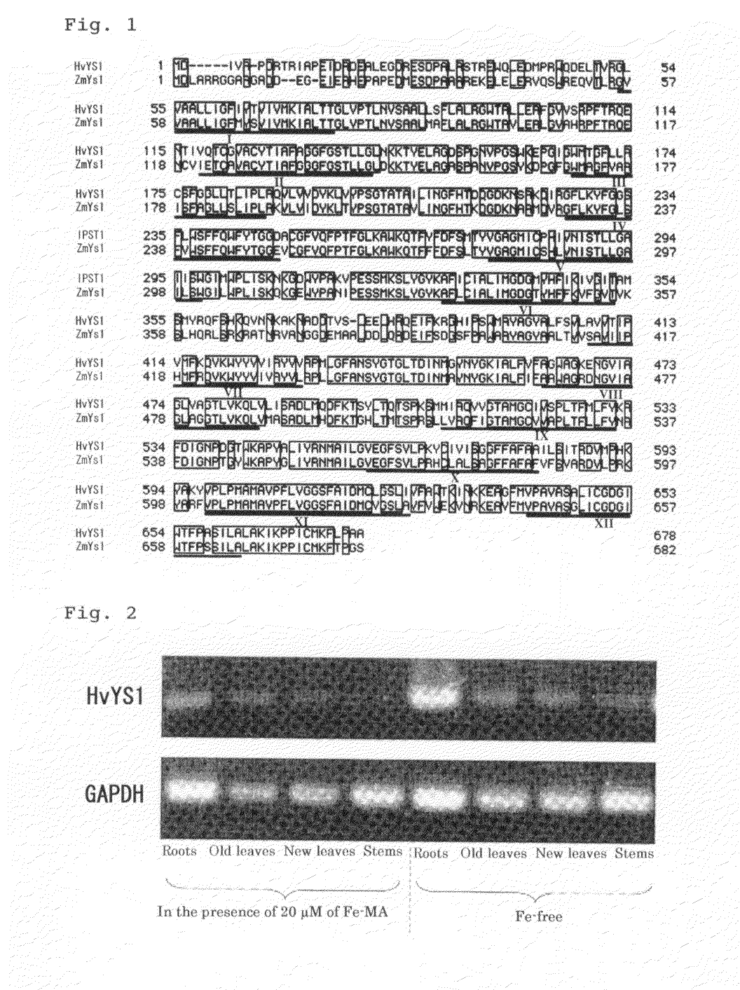

The amino acid sequence (SEQ ID NO: 2) of HvYS1 protein was determined from the cDNA sequence. The protein has an amino acid length of 678 with about 73% of homology with ZmYS1 protein of maize. Both proteins show particularly high homology in 12 membrane penetration regions of ZmYS1 predicted by SOSUI program (see FIG. 1).

### Example 2

### Comparison of Gene Expression Level in the Tissue of Barley

After seeding barley (Morex), sprouts of barley were pre-cultivated in a cultivation medium supplemented with 20 μM of mugineic acid-iron complex for 1 week. The plant was then cultivated on an iron-free cultivation medium or on a cultivation medium supplemented with 20 μM of mugineic acid-iron complex for 6 days, and RNA was extracted from the roots of barley in each medium. The extracted RNA was subjected to real time RT-PCR (26 cycles) using each of primers in Table 9 by ABI Prism 7000 Sequence Detection System (by Applied Biosystems Co.).

TABLE 9

| Primer | Base sequence | Sequence table |
|---|---|---|
| RT-PCR | 5′-AAAAAATGCGGACGACACTGT | SEQ ID NO: 22 |
| forward | | |
| primer | | |
| RT-PCR | 5′-AGGCATAACCAGCGTATGCC | SEQ ID NO: 23 |
| reverse | | |
| primer | | |

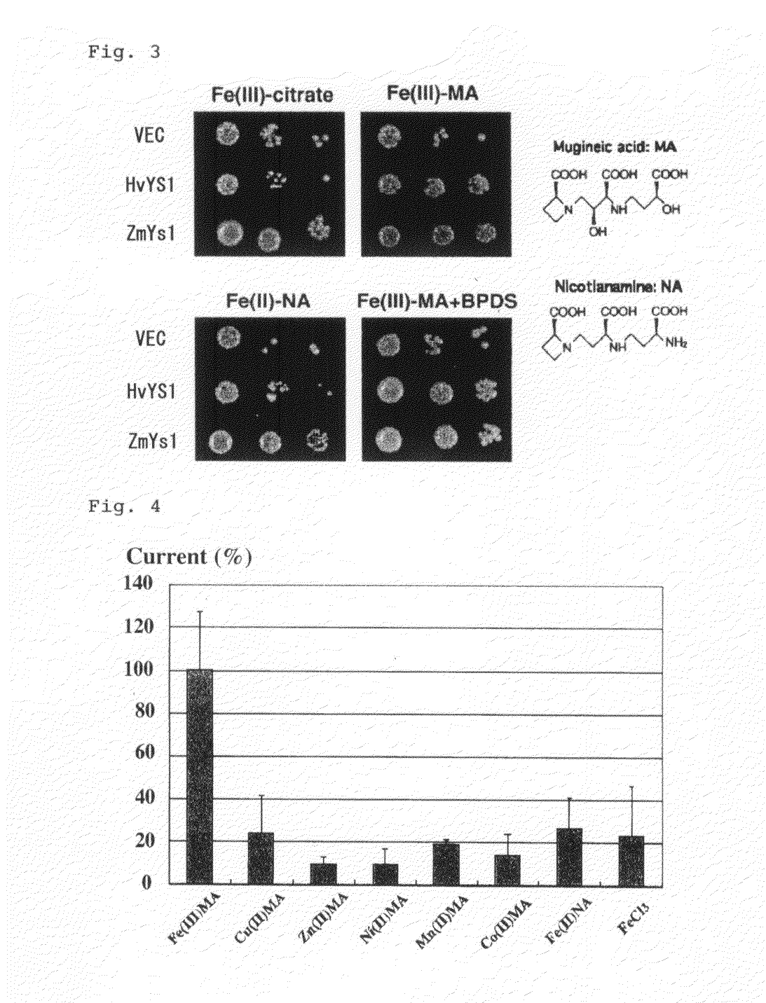

GAPDH (glyceraldehyde-3-phosphatedehydrogenase) gene was used as a control. It was found that while HvYS1 was seldom expressed when a mugineic acid-iron complex was abundant, the expression level increased selectively in the roots in an iron-deficient state (see FIG. 2).

### Example 3

### Function of HvYS1 in Transformed Yeast

Since double mutant fet3fet4 (DDY4 strain) of budding yeast (Saccharomyces cerevisiae) is defective in two genes responsible for absorption of divalent iron (fet3 (a gene for absorbing divalent iron after converting trivalent iron into divalent iron) and fet4 (a gene for absorbing divalent iron as it is)), the yeast can grow neither on an iron-limiting medium (Eide, D. et al., Proc. Natl. Acad. Sci. USA, 1996, vol. 93, pp. 5624-5628) nor by taking advantage of iron complexed with mugineic acid (Loulergue, C., Gene, 1998, vol. 225, pp. 47-57). To investigate the function of HvYS1 in iron transport, the present inventors have studied, using DDY4 strain into which HvYS1 cDNA has been introduced, whether the DDY4 strain in which the gene is expressed is able to grow on a medium containing Fe(III).MA as sole source of iron.

The following three plasmids were independently introduced into each of DDY4 strain and DY1457 (wild) strain: (1) a plasmid into which HvYS1 cDNA cloned at the NotI site of the expression vector pFL61 (by ATCC Co.) was inserted; (2) a plasmid into which ZmYS1 cDNA (Curie, C. et al., Nature, 2001, vol. 49, pp. 346-349) cloned in the same pFL61 vector was inserted; and (3) the pFL61 vector as a reference into which none of the above-mentioned genes was inserted.

Subsequently, the inventors conducted cultivation tests by mixing three different iron sources, or Fe(III).citrate, Fe(III).MA, and Fe(III).NA, with the medium in order to determine substrate selectivity, if any, of HvYS1. The yeast was cultivated in minimum media—Ura supplemented with 50 μM Fe(III).citrate, 10 μM Fe(III).MA, or 10 μM Fe(II).NA, and 10 μM FeCl₂ or FeCl₃ as a blank. Also, 10 μM of BPDS that is a potent chelating agent of divalent iron was added to the medium supplemented with 10 μM of Fe(III).MA in order to investigate whether the growth of the yeast is inhibited. Fe(III).MA was prepared according to von Wiren, N. et al. , Biochem. Biophys. Acta, 1998, vol. 1371, pp. 143-155. Nicotinamine was purchased from T. Hasegawa Co., and Fe(II).NA was prepared according to Schaaf, G. et al. , J. Biol. Chem., 2004, vol. 279, pp. 9091-9096. The yeast was cultivated at 30° C. for 4 days. Three solutions of the yeast culture (diluted to optical densities (OD) of 0.2, 0.02 and 0.002, respectively, at a wavelength of 600 nm) were spotted on a plate.

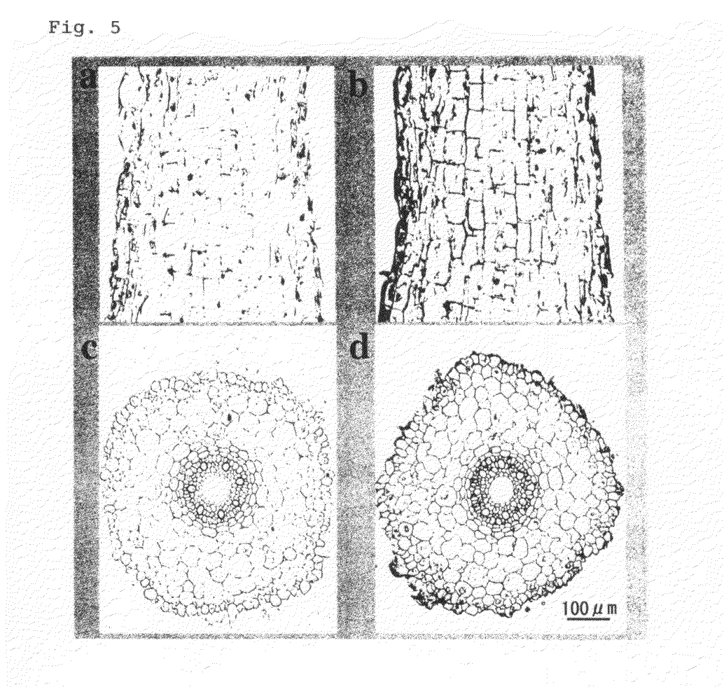

DDY4 strain expressing HvYS1 did not grow when FeCl₂, FeCl₃, or Fe(III).citrate was supplied as a sole iron source. In the presence of 10 μM of Fe(III).MA, DDY4 strain expressing HvYS1 grew at the same level as DDY4 strain expressing ZmYS1. When iron was supplied as Fe(III).MA chelate, DDY4 strain expressing HvYS1 could grow. However, when iron was supplied as Fe(III).citrate, the DDY4 strain could not grow or was strongly inhibited from growing. Accordingly, it was suggested that the HvYS1 protein encodes an iron transporter selective to Fe(III).MA. To elucidate this, BPDS as a potent Fe(II) chelating agent was added to the medium supplemented with Fe(III).MA so that remaining Fe(II) was completely removed from the medium. DDY4 strain expressing HvYS1 grew in the medium supplemented with Fe(III).MA in the presence of BPDS. This strongly suggests that the HvYS1 protein is a transporter protein of phytosiderophore-linked Fe(III). While ZmYS1, a transporter of maize transports Fe(II).NA that is present in the entire plant as well as Fe(III).MA, DDY4 strain expressing HvYS1 does not absorb Fe(II).NA, or growth was strongly inhibited. This shows that HvYS1 protein is contained in the roots, and selectively works for absorbing Fe(III).MA from soil (see FIG. 3).

### Example 4

### Action of HvYS1 in Electrophysiological Activity in Transformed Xenopus Oocyte Cell

HvYS1 cDNA was inserted into the XbaI and BamHI sites of pSP64Poly(A) vector (by Promega Co.), and cRNA was produced with mMESSAGE mMACHINE Kit (by Ambion Inc.) using the vector.

The abdomen of Xenopus (purchased from Hamamatsu Seibutsu Kyozai Co.) was incised, and Xenopus oocytes were extracted. The oocyte cells were put into a centrifuge tube having OR-2 solution (82.5 mM of NaCl, 2 mM of KCl, 1 mM of MgCl₂, and 5 mM of HEPES) containing 2 mg/mL of Collagenase Type IA (by Sigma Co.). After 2 hours' incubation at room temperature, the sample was washed three times with OR-2 solution and three times with ND-96 solution (96 mM of NaCl, 2 mM of KCl, 1 mM of MgCl₂, 1.8 mM of CaCl₂, and 5 mM of HEPES). 50 nL of cRNA (50 μg/mL) was injected into the Xenopus oocyte cells with a digital micro-dispenser (by Drummond Scientific Co.). The oocyte cells were cultivated at 17° C. in ND-96 solution for 48 to 72 hours.

Subsequently, the inventors have formed mugineic acid complexes of copper, zinc, nickel, manganese, and cobalt as substrates other than Fe(III).MA as in the case of iron in order to determine substrate selectivity, if any, of HvYS1 protein. The oocyte cells in which HvYS1 is expressed were set in a chamber filled with the ND-96 solution, and electrophysiological activity was measured after spraying 10 μL of each 5 mM substrate (final concentration of 50 μM). Two micro-electrodes filled with 3M KCl was inserted into the oocyte cell (internal resistance of 0.5 to 2 MO), and the voltage was clamped using Axoclamp type-2 dual electrode voltage clamp amplifier (by Axon Co.) in a mode in which the test vessel was clamped at 0 mV. The electric current was flowed through a 1 kHz low-path filter (−3 dB, 8 pole Bessel filter/cyber amplifier by Axon Co.), and sampled with a digital data 1200 interface (by Axon Co.) at 10 kHz. The sampled data was digitalized and stored. ORIGIN 6.1 software (by Microcal Software Co.) was used for programming and storage of voltage, and analysis of the recorded and stored data. The measurements were made at a fixed voltage of −60 mV.

Mugineic acid-iron(III) complex showed an overwhelmingly strong voltage change as compared with various mugineic acid-metal complexes other than Fe(III).MA and nicotianamine Fe(II) complex. The response to the nicotianamine Fe(II) complex showed good matching with the study results of yeast in Example 3 (FIG. 4).

### Example 5

### Expression Site of HvYS1 in the Roots of Barley

All the samples were manipulated in an RNase-free condition. The roots of barley in an iron-deficient state prepared in Example 1 were placed in 4% paraformaldehyde/PBS, and evacuation and resumption of the pressure were repeated for every 15 minutes until the roots were sunk. Then the sample was incubated at 4° C. for 24 hours. After being washed with PBS twice for 30 minutes each, the sample was incubated in 30%, 40%, 50% and 60% aqueous ethanol solution in series for 30 minutes each. The sample was incubated at 4° C. in 70% aqueous ethanol solution for 24 hours. The next day, the sample was dehydrated by sequentially immersing in 85%, 95%, and 100% aqueous ethanol solution, and then was sequentially transferred into 25%, 50%, 75% and 100% xylene/ethanol solutions. A paraffin chip (Paraplast Plus by Tyco Co.) was added to the 100% xylene, and the solution was incubated at 42° C. for 24 hours. Paraffin was exchanged twice a day, and the tissue was embedded in paraffin after incubating at 60° C. for 3 days. Contiguous 5 μm slices were prepared using a rotary microtome (by Ikemoto Scientific Technology Co., Ltd.), placed on an MAS-coat slide glass (by Matsunami Co.), and stored at −20° C.

A cRNA probe was prepared using a DIG (digoxigenin) RNA labeling kit (by Roche Co.) by introducing HvYS1 cDNA into the XbaI and HindIII sites of a plasmid vector pBluescript KS(+). The sense probe was prepared using T7 polymerase after linearizing the vector with HindIII restriction enzyme, while the antisense probe was prepared using T3 polymerase after linearizing the vector with XbaI restriction enzyme. The probes were fragmented into 150 by fragments by alkali treatment (at 60° C. for 56 minutes in a solution containing 42 mM NaHCO₃ and 63 mM Na₂CO₃), and the fragments were precipitated with ethanol and dissolved in DEPC treatment water.

In situ hybridization was performed according to the protocol by Cindy Lincoln and David Jackson. The slide with the paraffin slice was dried for 10 minutes, and was treated with xylene twice for 10 minutes each, with 100% ethanol and 90%, 80%, 70%, and 50% aqueous ethanol solution for 2 minutes, respectively, and with PBS twice for 5 minutes each.

Subsequently, the sample was treated with proteinase K (1 μg/mL proteinase K (by Sigma Co.), 100 mM Tris-HCl (pH 7.4), and 50 mM EDTA) at 37° C. for 30 minutes, washed with PBS for 2 minutes each, and fixed with 4% PFA/PBS for 20 minutes. The fixed sample was washed twice with PBS for 2 minutes, with 0.2N HCl for 10 minutes, with PBS twice for 2 minutes each, with PBS containing 2 mg/mL of glycine twice for 15 minutes and with PBS twice for 3 minutes each, and was acetylated. After washing with 2×SSC (150 mM NaCl, 15 mM sodium citrate, pH 7.4) twice for 2 minutes each, the acetylated sample was dehydrated until 100% ethanol was obtained as described above, and dried in a desiccator for 1 hour. A hybridization solution containing the probe (50% formamide in deionized water, 10 mM Tris-HCl (pH 7.4), 5 mM EDTA, 1× Denhat's solution, 10% (w/v) dextran sulfate, 20 μg/mL yeast tRNA, 0.3 M NaCl, and 0.3 M DDT (dithiothreitol)) was hybridized on the slice. The slice was covered with a paraffin film, and incubated at 50° C. for 16 hours. The sample was washed twice with 0.2×SSC at 55° C. for 60 minutes, and was treated with RNase (RNase A 20 μg/mL, 0.5 M NaCl, 10 mM Tris-HCl (pH 7.4), and 1 mM EDTA) at 37° C. for 30 minutes. The sample was then washed twice with 0.2×SSC at 55° C. for 30 minutes, and treated with PBS at room temperature for 5 minutes in order to permit DIG to develop a color. The sample was treated with buffer solution 1 (0.15 M NaCl, and 100 mM Tris-HCl (pH 7.4)) twice for 10 minutes each, with buffer solution 2 (15% (w/v) blocking reagent (by Roche Co.) /buffer solution 1) for 45 minutes, and with buffer solution 1 for 5 minutes, and then reacted with anti-DIG antibody (by Roche Co., 750-fold dilution) at room temperature for 1 hour. The sample was washed twice with buffer solution 1 for 5 minutes each, and with buffer solution 3 (0.1M NaCl, 100 mM Tris-HCl (pH 9.5), and 50 mM MgSO₄) for 10 minutes, and then made to develop a color by treating with an alkali phosphatase NBT/BCIP kit (by Nacalai Tesque Co.) overnight. The sample was treated with buffer solution 4 (10 mM Tris-HCl (pH 8.0), and 1 mM EDTA) for 10 minutes to stop color development, washed with distilled water, sealed in a crystal mount (by Cosmo Bio Co.), and then was observed with an optical microscope (Eclipse E400 by Nikon Corp.). The results are shown in FIG. 5. It was shown that color development was observed at epidermal cell portions of the roots of transgenic barley into which iron-deficiency antisense HvYS1 had been introduced, and HvYS1 was strongly expressed (FIG. 5).

### Example 6

### Creation of Transgenic Plant Into Which HvYS1 has Been Introduced

The molecular biological technique used this example was in accordance with the method described in WO 96/25500 or Molecular Cloning (Sambrook et. al., 1989, Cold Spring Harbor Laboratory Press) unless otherwise stated.

### (Construction of HvYS1 Expression Vector)

A DNA fragment (about 1.3 kb) was obtained by digesting pCGP 1394 (described in Tanaka et al., 1995, Plant Cell Physiol., 36: 1023-1031) with HindIII and SacII; a DNA fragment (about 2 kb) was obtained by digesting pCGP1394 with PstI, blunt-ending with a blunting kit (by Takara Bio Inc.), and digesting with SacII; and a DNA fragment (about 12 kb) was obtained by digesting pBinPLUS (van Engelen et al., 1955, Transgenic Research, 4, 288-290) with Sac I, blunt-ending, and digesting with HindIII. The three fragments were ligated to obtain plasmid pSPB185.

PCR products obtained by amplification of HvYS1 primers in Table 10 were sub-cloned to the vector of pCRII-TOPO vector using TOPO-TA cloning kit (by Invitrogen Co.)

TABLE 10
HvYS1 primer

| | | Sequence |
|---|---|---|
| Primer | Base sequence | table |
| Forward | 5′-GCTCTAGAATGGACATCGTCGCC-3′ | SEQ ID |
| primer | | NO: 24 |
| Reverse | 5′-CCCAAGCTTTTAGGCAGCAGGTAG-3′ | SEQ ID |
| primer | | NO: 25 |

The forward primer in Table 10 was obtained by adding XbaI sequence (GCTCTAGA) as a restriction enzyme site to the 5′-end of HvYS1 translation region, while the reverse primer was obtained by adding HindIII sequence (CCCAAGCTT) as a restriction enzyme site to the 3′-end of HvYS1 translation region.

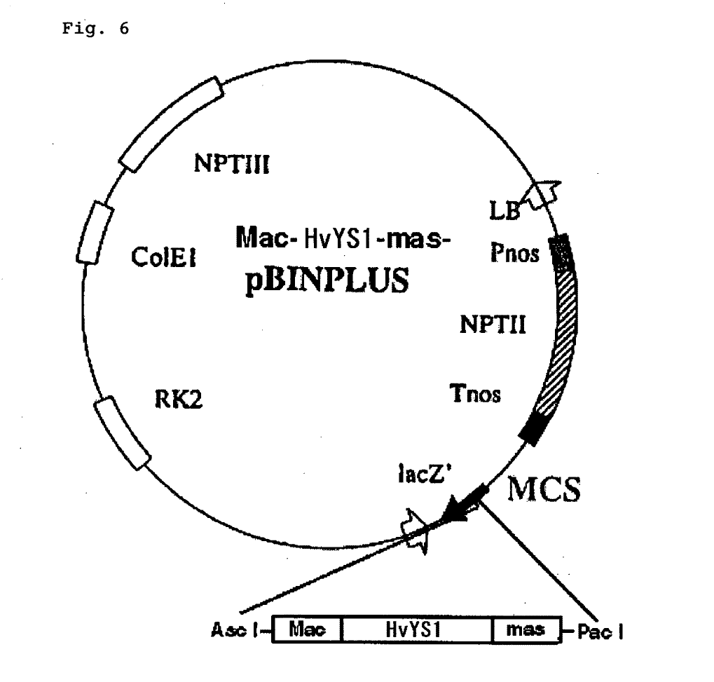

The plasmid (sub-cloned pCRII-TOPO vector) containing HvYS1 was firstly digested with HindIII, protruding ends were blunt-ended with a blunting kit (by Takara Bio Inc.), and the blunted fragments were further digested with XbaI to isolate DNA fragments (about 2 kb) containing HvYS1. Amplified pSPB185 was separately digested with KpnI, the ends were also blunt-ended, and blunted fragments were further digested with XbaI to obtain DNA fragments (about 14 kb). Then, the DNA fragment containing HvYS1 was ligated with the DNA fragment (about 14 kb) to produce plasmid Mac-HvYS1-mas-pBinPlus shown in FIG. 6. This plasmid is used for constructive expression of HvYS1 with Mac promoter (Comai et al., 1990, Plant Mol. Biol., 15, 373-381) in plants.

### (Transformation of Petunia)

Subsequently, agrobacterium (Agrobacterium tumefaciens strain AG 10) was transformed using Mac-HvYS1-mas-pBinPlus based on a known method (Plant J., 5, 81, 1994). Then, the transformed agrobacterium was infected to petunia (Petunia hybrid cultivar Saffinia Purple Mini (by Suntoryflowers Co., Ltd.)) to introduce the translation-region gene of HvYS1 into the petunia.

All the plants were kept at 23±2° C. with irradiation (60 μE, cold-white fluorescence lamp) for 16 hours. When the roots grew to a length of 2 to 3 cm, the transgenic petunia plant was transplanted into Debco 5140/2 pot mix (sterilized with an autoclave) in a 15 cm cultivation pot. 4 weeks later, the plant was re-transplanted into a 15 cm pot with the same pot mix, and kept at 23° C. with irradiation for 14 hours (300 μE, halogenated mercury lamp).

### (Detection of Introduced HvYS1 by RT-PCR Method)

The leaves of obtained transgenic petunia were mashed, and total RNA was extracted using RNeasy Plant Mini Kit (by Qiagen Co.). cDNA was prepared from 1 μg of extracted RNA with the First Strand cDNA Synthesis kit using the SuperScript™ II RT enzyme (by Invitrogen Co.). To confirm the presence of HvYS1, cDNA prepared from total RNA extracted from the transgenic petunia was used as a template, and was amplified by PCR using the forward primer (SEQ ID NO: 26 in the sequence table) and the reverse primer (SEQ ID NO: 27 in the sequence table) in Table 11. The forward primer synthesized an inner sequence from 889^{th} to 910^{th} from the HvYS1 base sequence (SEQ ID NO: 1 of the sequence table), and the reverse primer synthesized an inner sequence from 1644^{th} to 1621^{st} from the same base sequence. GADPH (glyceroaldehyde triphosphate dehydrogenase) gene was used as a control gene. The forward primer and the reverse primer in Table 12 were used as the primers of the GAPDH gene.

TABLE 11
HvYS1 primer

| | | Sequence |
|---|---|---|
| Primer | Base sequence | table |
| Forward | 5′-CAATGGTTCTACACTGGAGGCG-3′ | SEQ ID |
| primer | | NO: 26 |
| Reverse | 5′-CATCAAATCGGCAGAGATAAGCAC-3′ | SEQ ID |
| primer | | NO: 27 |

TABLE 12
Primer of control GAPDH

| | | Sequence |
|---|---|---|
| Primer | Base sequence | table |
| Forward | 5′-GGTCGTTTGGTTGCAAGAGT-3′ | SEQ ID NO: 28 |
| primer | | |
| Reverse | 5′-CTGGTTATTCCATTACAACTAC-3′ | SEQ ID NO: 29 |
| primer | | |

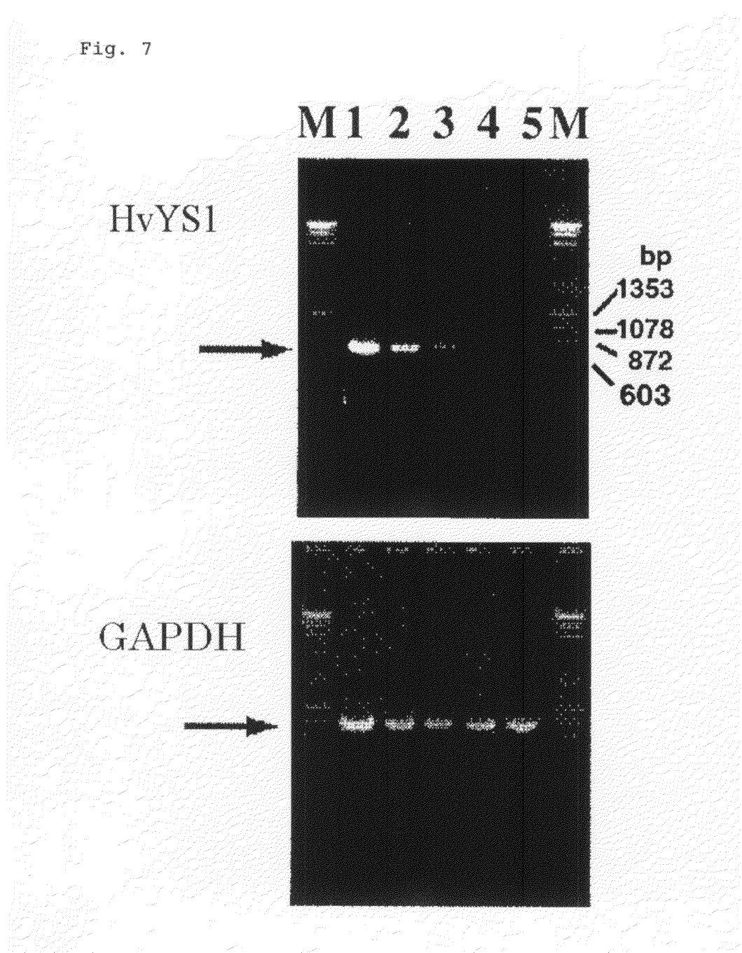

The PCR product was detected by 1.2 w/v % agarose gel electrophoresis (FIG. 7).

A band at 755 by predicted as the PCR product derived from HvYS1 gene was detected in the transgenic plants (1 to 3 in FIG. 7) into which HvYS1 had been introduced, although the amounts of the PCR product were different, and it was confirmed that HvYS1 gene had been introduced into petunia. In normal petunia (4 and 5 in FIG. 7: control) into which HvYS1 gene was not introduced, while a PCR product of GAPDH (about 1000 bp) was detected, the PCR product derived from HvYS1 gene was not detected.

### INDUSTRIAL APPLICABILITY

Since the plant into which the gene of the invention has been introduced can grow on alkaline soil that has been conventionally unable to grow plants, the invention makes it possible to produce plants on alkaline soil.

1. A gene containing DNA encoding a transporter protein for selectively absorbing mugineic acid-iron complex.

2. The gene according to claim 1, which is any one of (a) to (d) below:
(a) a gene comprising a DNA encoding a transporter protein having an amino acid sequence represented by SEQ ID NO: 2 in the sequence table;
(b) a gene comprising a DNA encoding a transporter protein having an amino acid sequence resulting from deletion, substitution, or addition of one or several amino acids in the amino acid sequence in (a), and having an activity for selectively a absorbing mugineic acid-iron complex;
(c) a gene comprising a DNA encoding a transporter protein having an amino acid sequence of which homology with the amino acid sequence in (a) is at least 60%, and having an activity for selectively absorbing mugineic acid-iron complex; and
(d) a gene comprising a DNA that hybridizes with the DNA in (a) under a stringent condition and encodes a transporter gene having an activity for selectively absorbing a mugineic acid-iron complex.

3. A vector characterized by containing the gene according to claim 1.

4. A host cell characterized by containing the vector according to claim 3.

5. A transgenic plant into which the gene according to claim 1 is introduced.

6. A transgenic plant into which the vector according to claim 3 is introduced.

7. A method for producing a transporter protein having an activity for selectively absorbing mugineic acid-iron complex characterized by cultivating the host cell according to claim 4 under a condition for expressing the gene.

8. A transporter protein having an activity for selectively absorbing mugineic acid-iron complex and produced by the method according to claim 7.

9. A protein, which is any one of (a) to (c) below having an activity for selectively absorbing a mugineic acid-iron complex:
(a) a protein comprising an amino acid sequence represented by SEQ ID NO: 2 in the sequence table;
(b) a protein comprising an amino acid sequence resulting from deletion, substitution, or addition of one or several amino acids in the amino acid sequence in (a), and having an activity for selectively absorbing a mugineic acid-iron complex; and
(c) a protein comprising an amino acid sequence of which homology with the amino acid sequence in (a) is at least 60%, and having an activity for selectively absorbing a mugineic acid-iron complex.

10. The RNA transcript of the DNA according to claim 1.

11. The transgenic plant according to claim 6 characterized by belonging to any family selected from the group consisting of Poaceae, Moraceae, Leguminosae, Rosaceae, Theaceae, Rubiaceae, Fagaceae, Rutaceae and Solanaceae.

12. A method for giving an activity for selectively absorbing mugineic acid-iron complex to a plant characterized by permitting the gene according to claim 1 to be expressed in the plant.


SEQUENCE LISTING (30 sequences)

SEQ ID NO: 1 - DNA, 2430 bp, Hordeum vulgare
  HvYS1
taatctcaccgcaaaagcacacggttccagctcgcctcgtgggcggaaacggggaacgacttcctggaagctgcggcctccagtgattcttcttccacggtacagtgatcagtcgaccactacgaccttgatcgttgagcagctgcggaggcaagaagcagagccaccatggacatcgtcgccccggaccgcacgcggatcgcgccggagatcgacagggacgaggccctggagggcgacagggagtcggacccggcgctggcgtcgacgcgcgagtggcagctggaggacatgccacggtggcaggacgagctgacggtgcggggcctggtggcggcgctgctcatcgggttcatctacaccgtcatcgtcatgaagatcgcgctcaccacggggctggtgcccacgctgaacgtctccgccgcgctgctctccttcctggcgctccgcgggtggacgcgcctgctggagcgcttcggcgtcgtgtcccgccccttcacgcggcaggagaacaccatcgtccagacctgcggcgtggcatgctacaccatcgcgtttgccggtgggttcgggtcaaccttgctgggcctgaacaagaagacgtacgagctggccggtgactcgccgggcaacgtgcccggaagctggaaggagccagggataggctggatgacggggttcctcctcgcttgcagcttcgggggcctcctcactttgattcccctgagacaggtactggtcgtcgactacaaattagtttacccaagtgggactgcaactgctattcttataaacgggttccataccgatcaaggggacaagaattcaagaaagcaaatccgtggattcctgaaatactttgggggtagctttctgtggagtttcttccaatggttctacactggaggcgatgcttgtggatttgttcagttcccaactttcggtttgaaggcctggaagcagacattctactttgactttagcatgacatacgttggtgccgggatgatttgtccacatatagtaaatatatctaccctccttggtgcaattatctcatggggaataatgtggccactcatcagtaaaaacaagggggattggtaccctgcaaaagtaccagaaagcagcatgaaaagtttgtacggttacaaggccttcatatgcatagctctcatcatgggggatggcatgtaccacttcataaaaattgttggcatcactgctatgagcatgtatcggcaatttagccacaagcaggttaacaacaaagcaaaaaatgcggacgacactgtctcgcttgaggagttacaccgccaggagatctttaagagaggccacatcccctcttggatggcatacgctggttatgccttgtttagcgttcttgcagtggttacaataccagtaatgttcaaacaagtgaagtggtactatgttgttatagcctatgtcgttgcccccatgcttggatttgccaattcatacgggacagggcttaccgacatcaacatgggctataactatggcaagatcgctctctttgtctttgcgggatgggccggtaaagagaatggtgtcattgccggccttgttgctggcaccttggttaagcagttggtgcttatctctgccgatttgatgcaagacttcaagacgagttacctcactcaaacatcaccaaaatccatgatgattgcacaagttgttggaacagccatgggttgcattgtctcccccctcacgttcatgctcttctacaaggcatttgatattggtaacccagatggtacttggaaggcaccttatgcactcatataccgcaatatggcaatacttggtgtggagggcttctcggtgttgccgaagtattgcatcgtgatatccggtggatttttcgcctttgcggcgattttaagtataacaagagatgtcatgccccacaagtatgcgaagtatgtgcccctgccaatggcaatggcagttccattccttgtaggtgggagctttgctattgatatgtgcctcgggagtttgatagtttttgcatggaccaagataaacaagaaggaggctggcttcatggtgcctgcggttgcatccgctttgatatgtggggatggcatatggacgttccctgcttccattcttgctcttgccaagattaaaccaccaatttgcatgaagtttctacctgctgcctaacggaccgaaaaatatactaggtgaccaaagatgcattgaatttccatagttgtgccttgctgaactatcttttagtgtttgtttactggagatgcttcggtactcgagtgtctaggaatgatacgttggtatgtgcttttacgggtataacctgaattttcttattttatgaatctggggtatacttatcagttgctatgagtatggatctaatgatttggctga

SEQ ID NO: 2 - protein, 678 aa, Hordeum vulgare
  Iron(III)-phytosiderophore transporter protein
MDIVAPDRTRIAPEIDRDEALEGDRESDPALASTREWQLEDMPRWQDELTVRGLVAALLIGFIYTVIVMKIALTTGLVPTLNVSAALLSFLALRGWTRLLERFGVVSRPFTRQENTIVQTCGVACYTIAFAGGFGSTLLGLNKKTYELAGDSPGNVPGSWKEPGIGWMTGFLLACSFGGLLTLIPLRQVLVVDYKLVYPSGTATAILINGFHTDQGDKNSRKQIRGFLKYFGGSFLWSFFQWFYTGGDACGFVQFPTFGLKAWKQTFYFDFSMTYVGAGMICPHIVNISTLLGAIISWGIMWPLISKNKGDWYPAKVPESSMKSLYGYKAFICIALIMGDGMYHFIKIVGITAMSMYRQFSHKQVNNKAKNADDTVSLEELHRQEIFKRGHIPSWMAYAGYALFSVLAVVTIPVMFKQVKWYYVVIAYVVAPMLGFANSYGTGLTDINMGYNYGKIALFVFAGWAGKENGVIAGLVAGTLVKQLVLISADLMQDFKTSYLTQTSPKSMMIAQVVGTAMGCIVSPLTFMLFYKAFDIGNPDGTWKAPYALIYRNMAILGVEGFSVLPKYCIVISGGFFAFAAILSITRDVMPHKYAKYVPLPMAMAVPFLVGGSFAIDMCLGSLIVFAWTKINKKEAGFMVPAVASALICGDGIWTFPASILALAKIKPPICMKFLPAA

SEQ ID NO: 3 - DNA, 2049 bp, Zea mays
  ZmYS1
atggaccttgcacggagaggcggtgccgcaggcgcggacgacgagggggagatcgagaggcacgagccggcgcccgaggacatggagtccgaccccgcagcggcgcgcgagaaggagctggagctggagcgggtgcagtcgtggcgggagcaggtgactctgcgcggcgtggtggcggcgctgctgatcggcttcatgtacagcgtgatcgtgatgaagatcgcgctcaccacggggctggtgcccacgctgaacgtctccgcggcgctgatggcgttcctggcgctccgcgggtggacgcgcgtgctggagcgcctcggcgtggcgcaccgccccttcacgcgccaggagaactgcgtcatcgagacctgcgccgtcgcgtgctacaccatcgcgttcggcggtgggttcggctccacgctgctgggcctggacaagaagacgtacgagctggccggggcctcgccggccaacgttccgggcagctacaaggaccctgggttcggctggatggccggattcgtcgcggcgatcagcttcgccggcctcctaagcctgatccccctcagaaaggttctggtcattgactacaagctaacttacccaagcgggactgcgaccgctgttctcataaacgggttccacaccaagcaaggagacaagaacgcaaggatgcaagtccgagggttcctcaagtactttgggctcagcttcgtgtggagctttttccagtggttctacacaggcggtgaagtttgcggctttgttcagtttcctacgttcggtctgaaggcctggaagcagacgttcttctttgattttagcctcacgtacgttggtgcggggatgatctgttcgcacctcgtgaacatctccaccctccttggtgccatcctgtcatgggggatactgtggccactcatcagcaagcagaaaggggagtggtaccctgcgaacatacctgagagtagcatgaaaagcttatacggttacaaggccttcctctgcatagctctgatcatgggagacggtacataccacttctttaaagtcttcggtgtcactgttaagagtctgcatcaacggctgagccgcaaacgtgctaccaacagagtggcaaacggtggagacgaaatggccgcgcttgacgacctacagcgtgacgagatcttcagcgacgggtctttccccgcctgggcagcttacgccgggtacgcggcgctgaccgtcgtctcagcggtcatcatcccgcacatgttccggcaggtcaagtggtactacgtgatcgtggcctacgtcctcgcccctctcctcggcttcgccaactcctacggcacggggctcaccgacatcaacatggcctacaactacggcaagatcgcgctcttcatcttcgcggcctgggccggcagggacaacggcgtcatcgcgggcctcgccggcggcaccctggtgaagcagctggtgatggcgtccgcggacctgatgcacgacttcaagacgggccacctgaccatgacgtcgcccaggtccctgctcgtggcgcagttcatcgggacggccatgggctgcgtcgtcgcgcccctcacgttcctgctcttctacaacgcgttcgacatcgggaaccccaccgggtactggaaggcgccgtacggcctcatctaccgcaacatggcgatcctcggcgtggagggcttctccgtgctgcccaggcactgcctcgcgctctccgctgggttcttcgccttcgccttcgtcttcagcgtcgcccgggacgtcctgccgcggaagtacgccaggttcgtgcccctgcccatggccatggccgtgccgttcctcgtgggcgggagcttcgcgatcgatatgtgcgtcgggagcctggccgtctttgtctgggagaaggtgaacaggaaggaggccgtgttcatggtgcctgcggttgcgtccggtttgatctgtggagacggcatatggaccttcccgtcttccattctcgctctggccaagatcaagccaccgatttgcatgaagttcactcctggaagctag

SEQ ID NO: 4 - DNA, 20 bp, Artificial Sequence
  Description of Artificial Sequence Synthetic primer
cattgccggccttgttgctg

SEQ ID NO: 5 - DNA, 19 bp, Artificial Sequence
  Description of Artificial Sequence Synthetic primer
cggccttgttgctggcacc

SEQ ID NO: 6 - DNA, 17 bp, Artificial Sequence
  Description of Artificial Sequence Synthetic primer
caggaaacagctatgac

SEQ ID NO: 7 - DNA, 16 bp, Artificial Sequence
  Description of Artificial Sequence Synthetic primer
gtaaaacgacggccag

SEQ ID NO: 8 - DNA, 19 bp, Artificial Sequence
  Description of Artificial Sequence Synthetic primer
ccacaagcatcgcctccag

SEQ ID NO: 9 - DNA, 21 bp, Artificial Sequence
  Description of Artificial Sequence Synthetic primer
catcgcctccagtgtagaacc

SEQ ID NO: 10 - DNA, 20 bp, Artificial Sequence
  Description of Artificial Sequence Synthetic primer
cagtgtagaaccattggaag

SEQ ID NO: 11 - DNA, 18 bp, Artificial Sequence
  Description of Artificial Sequence Synthetic primer
gaatagcagttgcagtcc

SEQ ID NO: 12 - DNA, 21 bp, Artificial Sequence
  Description of Artificial Sequence Synthetic primer
gtagtcgacgaccagtacctg

SEQ ID NO: 13 - DNA, 20 bp, Artificial Sequence
  Description of Artificial Sequence Synthetic primer
cgaccagtacctgtctcagg

SEQ ID NO: 14 - DNA, 20 bp, Artificial Sequence
  Description of Artificial Sequence Synthetic primer
gaataatgaggccactcatc

SEQ ID NO: 15 - DNA, 20 bp, Artificial Sequence
  Description of Artificial Sequence Synthetic primer
ggctataacaacatagtacc

SEQ ID NO: 16 - DNA, 20 bp, Artificial Sequence
  Description of Artificial Sequence Synthetic primer
gcacacggttccagctcgcc

SEQ ID NO: 17 - DNA, 21 bp, Artificial Sequence
  Description of Artificial Sequence Synthetic primer
gatagttcagcaaggcacaac

SEQ ID NO: 18 - DNA, 20 bp, Artificial Sequence
  Description of Artificial Sequence Synthetic primer
cctccagtgattcttcttcc

SEQ ID NO: 19 - DNA, 21 bp, Artificial Sequence
  Description of Artificial Sequence Synthetic primer
gatagttcagcaaggcacaac

SEQ ID NO: 20 - DNA, 20 bp, Artificial Sequence
  Description of Artificial Sequence Synthetic primer
cctcctcgcttgcagcttcg

SEQ ID NO: 21 - DNA, 20 bp, Artificial Sequence
  Description of Artificial Sequence Synthetic primer
ggtgccagcaacaaggccgg

SEQ ID NO: 22 - DNA, 21 bp, Artificial Sequence
  Description of Artificial Sequence Synthetic primer
aaaaaatgcggacgacactgt

SEQ ID NO: 23 - DNA, 20 bp, Artificial Sequence
  Description of Artificial Sequence Synthetic primer
aggcataaccagcgtatgcc

SEQ ID NO: 24 - DNA, 23 bp, Artificial Sequence
  Description of Artificial Sequence Synthetic primer
gctctagaatggacatcgtcgcc

SEQ ID NO: 25 - DNA, 24 bp, Artificial Sequence
  Description of Artificial Sequence Synthetic primer
cccaagcttttaggcagcaggtag

SEQ ID NO: 26 - DNA, 22 bp, Artificial Sequence
  Description of Artificial Sequence Synthetic primer
caatggttctacactggaggcg

SEQ ID NO: 27 - DNA, 24 bp, Artificial Sequence
  Description of Artificial Sequence Synthetic primer
catcaaatcggcagagataagcac

SEQ ID NO: 28 - DNA, 20 bp, Artificial Sequence
  Description of Artificial Sequence Synthetic primer
ggtcgtttggttgcaagagt

SEQ ID NO: 29 - DNA, 22 bp, Artificial Sequence
  Description of Artificial Sequence Synthetic primer
ctggttattccattacaactac

SEQ ID NO: 30 - protein, 682 aa, Zea mays
MDLARRGGAAGADDEGEIERHEPAPEDMESDPAAAREKELELERVQSWREQVTLRGVVAALLIGFMYSVIVMKIALTTGLVPTLNVSAALMAFLALRGWTRVLERLGVAHRPFTRQENCVIETCAVACYTIAFGGGFGSTLLGLDKKTYELAGASPANVPGSYKDPGFGWMAGFVAAISFAGLLSLIPLRKVLVIDYKLTYPSGTATAVLINGFHTKQGDKNARMQVRGFLKYFGLSFVWSFFQWFYTGGEVCGFVQFPTFGLKAWKQTFFFDFSLTYVGAGMICSHLVNISTLLGAILSWGILWPLISKQKGEWYPANIPESSMKSLYGYKAFLCIALIMGDGTYHFFKVFGVTVKSLHQRLSRKRATNRVANGGDEMAALDDLQRDEIFSDGSFPAWAAYAGYAALTVVSAVIIPHMFRQVKWYYVIVAYVLAPLLGFANSYGTGLTDINMAYNYGKIALFIFAAWAGRDNGVIAGLAGGTLVKQLVMASADLMHDFKTGHLTMTSPRSLLVAQFIGTAMGCVVAPLTFLLFYNAFDIGNPTGYWKAPYGLIYRNMAILGVEGFSVLPRHCLALSAGFFAFAFVFSVARDVLPRKYARFVPLPMAMAVPFLVGGSFAIDMCVGSLAVFVWEKVNRKEAVFMVPAVASGLICGDGIWTFPSSILALAKIKPPICMKFTPGS